(19) United States
(12) Patent Application Publication
(10) Pub. No.: US 2025/0362362 A1
(43) Pub. Date: Nov. 27, 2025

(54) MAGNETIC RESONANCE IMAGING SUPPORT METHOD AND MAGNETIC RESONANCE IMAGING APPARATUS

(71) Applicant: CANON MEDICAL SYSTEMS CORPORATION, Tochigi (JP)

(72) Inventors: Hong YANG, Beijing (CN); Yutaka HOSHIYAMA, Nasushiobara (JP); Chunqi WANG, Beijing (CN)

(73) Assignee: CANON MEDICAL SYSTEMS CORPORATION, Tochigi (JP)

(21) Appl. No.: 19/193,272

(22) Filed: Apr. 29, 2025

(30) Foreign Application Priority Data
May 23, 2024 (CN) 202410651676.0
Mar. 11, 2025 (JP) 2025-038068

(51) Int. Cl. G01R 33/54; A61B 5/00; A61B 5/055; G01R 33/30

(52) U.S. Cl. CPC G01R 33/543; A61B 5/0037; A61B 5/055; G01R 33/307

(57) ABSTRACT

A magnetic resonance imaging support method according to an embodiment includes an acquisition step, a multi-planar reconstruction step, a first determination step, and a second determination step. The acquisition step acquires, by performing a scout scan on a subject, a plurality of first slice images corresponding to different slice positions, the plurality of first slice images corresponding to either sagittal planes or coronal planes. The multi-planar reconstruction step generates a second slice image corresponding to the axial plane by performing multi-planar reconstruction based on the plurality of first slice images. The first determination step determines a position corresponding to a specific part of the subject based on the second slice image. The second determination step determines a range to be scanned in a main scan based on the plurality of first slice images at the position determined by the first determination step.

### CROSS-REFERENCE TO RELATED APPLICATIONS

This application is based upon and claims the benefit of priority from Chinese Patent Application No. 202410651676.0, filed on May 23, 2024; and Japanese Patent Application No. 2025-038068, filed on Mar. 11, 2025, the entire contents of all of which are incorporated herein by reference.

### FIELD

Embodiments described herein relate generally to a magnetic resonance imaging support method and a magnetic resonance imaging apparatus.

### BACKGROUND

In recent years, the technology of whole-body magnetic resonance imaging using a magnetic resonance imaging apparatus has been attracting attention. In whole-body magnetic resonance imaging, a scan performed on the whole body of a subject is configured with a plurality of sub-scans. In each sub-scan, the scan is performed by moving the couch with the subject placed thereon such that a specific part of the subject is positioned in a scan area of a magnetic resonance imaging apparatus. Then, by repeatedly moving the couch and performing the scan, partial images of a plurality of continuous parts of the subject are acquired. After all partial images regarding the subject are acquired, each of the partial images is combined in the couch moving direction to generate a whole-body image of the subject.

In order to avoid degradation in the accuracy of images and generation of incomplete images caused when the subject is shifted from the center of scan at the time of performing a magnetic resonance imaging scan, it is common to first perform a scout scan to generate a scout image of the subject before performing the main scan, and to determine a scan plan for the main scan based on the scout image. Note here that a scout image is generated by scanning the sagittal plane or coronal plane of the subject in order to shorten the scan time for the scout scan.

In the case of whole-body magnetic resonance imaging, a scout scan is performed on the whole body of the subject to generate a whole-body scout image of the subject, when determining a scan plan for the main scan. Conventionally, a scan plan for the main scan in whole-body magnetic resonance imaging is generated manually by an operator, which is time consuming and labor intensive. In determining a scan plan for the whole-body magnetic resonance imaging, it is particularly time consuming and labor intensive to determine the start position and end position of the scan in the top-and-bottom direction of the subject as well as to determine the mid-axis lines of the scans in the left-and-right and front-and-rear directions of the subject. For specifying the start position and end position of the scan, the operator needs to manually specify the positions of the head top, the head center, knees, feet, and the like of the subject based on the whole-body scout image. In addition, for specifying the mid-axis line of the scan, the operator needs to manually mark the mid-axis line of the subject based on the whole-body scout image.

To address these issues, technologies for supporting generation of scan plans is proposed in which object detection algorithms and the like are used to extract positional information from scout images. For example, there is a known technology for supporting determining a scan plan for the main scan in the whole-body magnetic resonance imaging by detecting feature points (landmarks) of the subject from a whole-body coronal scout image (for example, Whole-body Dot engine by Siemens). However, since this technology uses a single slice image as a scout image, the positions of detected landmarks depend on the slice positions of the scout image, so that positioning of landmarks becomes inaccurate and inappropriate scan plans may be generated.

### BRIEF DESCRIPTION OF THE DRAWINGS

FIG. 1 is a diagram illustrating a configuration example of a magnetic resonance imaging apparatus according to a first embodiment;

FIG. 2 is a flowchart illustrating a magnetic resonance imaging support method according to the first embodiment;

FIG. 3 is a diagram illustrating examples of partial slice image stacks;

FIG. 4 is a diagram illustrating an example of a whole-body slice image stack;

FIG. 5 is a schematic diagram illustrating an example when generating whole-body slice images in axial planes based on a whole-body slice image stack in sagittal planes;

FIG. 6 is a schematic diagram illustrating a result of object detection performed on the whole-body slice images in the plurality of axial planes illustrated in FIG. 5;

FIG. 7 is a schematic diagram illustrating rough human body segments in whole-body slice images in the sagittal planes, each whole-body slice image being set based on the whole-body slice images in the axial planes;

FIG. 8 is a schematic diagram illustrating a method for specifying the positions of the head top and the head center based on the whole-body slice image stacks in the axial planes and the sagittal planes;

FIG. 9 is a schematic diagram illustrating a method for specifying the position of the knee center based on the whole-body slice image stacks in the axial planes and the coronal planes;

FIG. 10 is a schematic diagram illustrating a method for specifying the position of the spine based on the whole-body slice image stacks in the axial planes, the sagittal planes, and the coronal planes;

FIG. 11 is a diagram illustrating an example of a scan plan for a main scan displayed on a display unit;

FIG. 12 is a diagram for describing the issues of conventional magnetic resonance imaging support methods; and

FIG. 13 is a flowchart illustrating a magnetic resonance imaging support method according to a second embodiment.

### DETAILED DESCRIPTION

A magnetic resonance imaging support method according to an embodiment includes an acquisition step, a multi-planar reconstruction step, a first determination step, a second determination step, and a main scan step. The acquisition step acquires, by performing a scout scan on a subject, a plurality of first slice images corresponding to different slice positions, the plurality of first slice images corresponding to either sagittal planes or coronal planes. The multi-planar reconstruction step generates a second slice image corresponding to an axial plane by performing multi-planar reconstruction based on the plurality of first slice images. The first determination step determines a position corresponding to a specific part of the subject based on the second slice image. The second determination step determines a range to be scanned in a main scan based on the plurality of first slice images at the position determined by the first determination step. The main scan step performs a scan on the subject in the range determined by the second determination step.

### First Embodiment

A first embodiment relates to a magnetic resonance imaging support method and a magnetic resonance imaging apparatus. Hereinafter, the magnetic resonance imaging support method and the magnetic resonance imaging apparatus according to the first embodiment will be described with reference to the accompanying drawings.

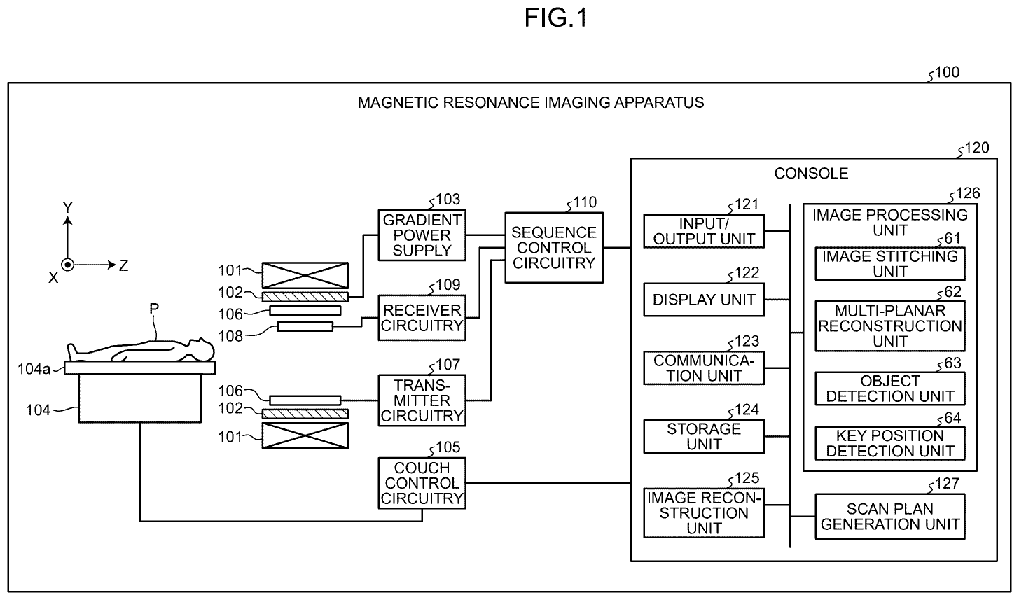

FIG. 1 is a diagram illustrating a configuration example of a magnetic resonance imaging apparatus 100 according to the first embodiment. The magnetic resonance imaging apparatus 100 includes a static magnetic field magnet 101, a static magnetic field power supply (not illustrated), a gradient coil 102, a gradient power supply 103, a couch 104, a couch control circuitry 105, a transmitter coil 106, transmitter circuitry 107, a receiver coil 108, receiver circuitry 109, a sequence control circuitry 110, and a console 120. The magnetic resonance imaging apparatus 100 further includes a gantry (not illustrated) that functions as a support unit for the static magnetic field magnet 101, the gradient coil 102, the transmitter coil 106, the receiver coil 108, and the like.

The static magnetic field magnet 101 is a magnet formed in a hollow and substantially cylindrical shape and generates a static magnetic field in the interior space. The static magnetic field magnet 101 is a superconducting magnet or the like, for example, which is excited by receiving a current supplied from the static magnetic field power supply. The static magnetic field power supply supplies a current to the static magnetic field magnet 101. As another example, the static magnetic field magnet 101 may be a permanent magnet, in which case the magnetic resonance imaging apparatus 100 may not include a static magnetic field power supply. A static magnetic field power supply may also be provided separately from the magnetic resonance imaging apparatus 100.

The gradient coil 102 is a coil formed in a hollow and substantially cylindrical shape, and placed on the inner side of the static magnetic field magnet 101. The gradient coil 102 is formed by combining three coils corresponding to the X, Y, and Z axes orthogonal to each other, and the three coils individually receive a current supplied from the gradient power supply 103 to generate a gradient magnetic field whose magnetic field intensity changes along each of the X, Y, and Z axes. Note that the Z-axis direction is the same direction as the static magnetic field, the Y-axis direction is the vertical direction, and the X-axis direction is the direction perpendicular to the Z axis and Y axis.

The gradient power supply 103 supplies a current to the gradient coil 102 under the control of the sequence control circuitry 110.

The couch 104 includes a couchtop 104a on which a subject P is placed, and inserts the couchtop 104a with the subject P placed thereon into a cavity of the gradient coil 102 under the control of the couch control circuitry 105.

The transmitter coil 106 is placed on the inner side of the gradient coil 102, and generates a high-frequency magnetic field by receiving RF pulses from the transmitter circuitry 107.

The transmitter circuitry 107 supplies the RF pulses corresponding to Larmor frequencies to the transmitter coil 106. The Larmor frequency is determined in accordance with the type of target atom and the magnetic field intensity.

The receiver coil 108 is placed on the inner side of the gradient coil 102, and receives magnetic resonance signals emitted from the subject P under the influence of the high-frequency magnetic field. Upon receiving the magnetic resonance signals, the receiver coil 108 outputs the received magnetic resonance signals to the receiver circuitry 109. Note that the transmitter coil 106 and the receiver coil 108 may be configured with a single coil that has transmitter and receiver functions.

The receiver circuitry 109 detects the magnetic resonance signals output from the receiver coil 108, and generates k-space data based on the detected magnetic resonance signals. Specifically, the receiver circuitry 109 performs analog-to-digital conversion on the analog magnetic resonance signals output from the receiver coil 108 to generate k-space data. The receiver circuitry 109 then transmits the generated k-space data to the sequence control circuitry 110. The receiver circuitry 109 may be placed on the gantry side where the static magnetic field magnet 101, the gradient coil 102, and the like are installed.

The sequence control circuitry 110 executes a scan on the subject P by driving the gradient power supply 103, the transmitter circuitry 107, and the receiver circuitry 109 according to the sequence information transmitted from the console 120, and transmits the scanned k-space data to the console 120. In the sequence information, the intensity of the current supplied from the gradient power supply 103 to the gradient coil 102, the timing of supplying the current, the intensity of the RF pulse supplied from the transmitter circuitry 107 to the transmitter coil 106, the timing of applying the RF pulse, the timing at which the receiver circuitry 109 detects the magnetic resonance signal, and the like are defined. The sequence control circuitry 110 is configured with, for example, an integrated circuit such as an application specific integrated circuit (ASIC) or a field programmable gate array (FPGA), or an electronic circuit such as a central processing unit (CPU) or a micro processing unit (MPU).

The console 120 includes an input/output unit 121, a display unit 122, a communication unit 123, a storage unit 124, an image reconstruction unit 125, an image processing unit 126, and a scan plan generation unit 127. The input/output unit 121, the display unit 122, the communication unit 123, the storage unit 124, the image reconstruction unit 125, the image processing unit 126, and the scan plan generation unit 127 are connected to each other via a bus.

The input/output unit 121 includes an input device and an input/output interface. The input device receives input operations from the user. The input/output interface inputs signals based on the received input operations to the console 120. The input device is, for example, a mouse, a keyboard, a trackball, switches, buttons, a joystick, a touch panel, a microphone, or the like. The input/output interface is, for example, a data transmission interface such as an optical fiber, USB, Thunderbolt, or the like. Furthermore, the input/output interface may be connected to a storage device or the like as an output device, and reads and writes various kinds of data to and from the storage device. The storage device is, for example, a hard disk drive (HDD), a solid state drive (SSD), or the like.

The display unit 122 includes a display device and a display interface. The display device displays information for the user and displays a user interface for the user to input information. The user interface is a graphical user interface (GUI) or the like, for example. The display interface transmits data to the display device to display images. The display device is, for example, a liquid crystal display (LCD), an organic electroluminescence (EL) display, or the like. The display interface is, for example, a video output interface such as a Digital Visual Interface (DVI) or a High-Definition Multimedia Interface (HDMI (registered trademark)).

The communication unit 123 connects the console 120, the couch control circuitry 105, the sequence control circuitry 110, and a remote device such as a server (not illustrated), and it is capable of transmitting and receiving various kinds of data to and from each of the devices. The communication unit 123 is configured with, for example, a wireless network adapter such as an IEEE 802.11/Wi-Fi adapter, an adapter for communicating with 3G, 4G/LTE, 5G networks, or the like, and a wired network adapter such as an optical fiber adapter, a power line adapter, or the like.

The storage unit 124 stores k-space data that is data acquired in magnetic resonance imaging, reconstructed image data, and the like. The storage unit 124 also stores various kinds of parameters used in the magnetic resonance imaging support method described later. In addition, the storage unit 124 stores parameters of a neural network. The storage unit 124 may also store various kinds of computer programs used by the console 120. The storage unit 124 is implemented by a storage device such as a read only memory (ROM), a flash memory, a random access memory (RAM), a hard disc drive (HDD), a solid state drive (SSD), a register, or the like.

The flash memory, HDD, and SSD are non-volatile storage media. These non-volatile storage media may be implemented by other storage devices connected via a network such as a network attached storage (NAS), an external storage server, and the like. Note that the above-mentioned network includes, for example, the Internet, wide area network (WAN), local area network (LAN), carrier network, dedicated line, and the like.

The image reconstruction unit 125 generates image data from the scanned k-space data by using a reconstruction algorithm based on the Fourier transform. Image data generated by the image reconstruction unit 125 is two-dimensional slice image data that indicates the structure of a specific slice position in the subject P. The slice position is the position on the plane where the cross section inside the subject is at. Note that the reconstruction algorithm for reconstructing image data is not limited, and the image reconstruction unit 125 may reconstruct image data using any reconstruction algorithms.

The image processing unit 126 performs image processing on the image data generated by the image reconstruction unit 125, and calculates information for specifying the scan parameters for the main scan. The image processing unit 126 includes an image stitching unit 61, a multi-planar reconstruction unit 62, an object detection unit 63, and a key position detection unit 64. The image stitching unit 61 generates a whole-body slice image stack in either the sagittal planes or the coronal planes based on a plurality of partial slice image stacks described later. Based on the data of the whole-body slice image stack in either the sagittal planes or the coronal planes generated by the image stitching unit 61, the multi-planar reconstruction unit 62 reconstructs a whole-body slice image stack in the axial planes and a whole-body slice image stack in the others of the sagittal planes and coronal planes. The object detection unit 63 performs object detection in the slice images included in the reconstructed whole-body slice image stack in the axial planes to detect information for generating a scan plan for the main scan. The key position detection unit 64 detects the position of a specific part of the human body as a key position based on the whole-body slice image stacks in the sagittal planes, the coronal planes, and the axial planes. The key position is the information for generating a scan plan for the main scan.

The scan plan generation unit 127 generates a scan plan for the main scan based on the information calculated by the image processing unit 126 and input of the user. A scan plan is configured with a plurality of scan parameters for performing a magnetic resonance imaging scan. Note here that the scan parameters are divided into two types that are: first type scan parameters that do not need to be determined based on a scout image and can be set in advance; and second type scan parameters that need to be determined based on the scout image.

The first type scan parameters include scan sequence type, inspection range, field of view (FOV), couch moving distance, matrix, slice thickness, slice gap, and the like. The scan sequence type determines the effect of the display of various kinds of tissues in the images acquired by magnetic resonance imaging. Each type of scan sequence is applied to observe different tissues. A scan sequence includes, for example, T1-weighted imaging, T2-weighted imaging, fluid attenuated inversion recovery (FLAIR), diffusion weighted magnetic imaging, and the like. The inspection range is the range of the part of the subject P that needs to be examined. The inspection range may be, for example, the whole body, the spine, or the like. FOV is the size of the area that can be examined by each sub-scan included in a whole-body magnetic resonance scan, and it is the physical size of the image to be generated. The couch moving distance is the distance of the couch moved between the sub-scans included in a whole-body magnetic resonance scan. The matrix, slice thickness, and slice gap are used for determining the spatial resolution of images acquired by magnetic resonance imaging.

The second type scan parameters include the slice direction, the start position and end position of a whole-body magnetic resonance scan, the number of times of sub-scans, the positions of the sub-scans, and the like. The slice direction includes coronal, sagittal, and axial directions, and determines the direction of the scanned slice images. The start position and end position of a whole-body magnetic resonance scan are the start point and end point of the scan in the top-and-bottom direction of the subject P. The start point of the scan is set at the head top, the head center, or the like, for example, and the end point of the scan is set at the knee center, feet, or the like, for example. The number of times of sub-scans is the number of sub-scans included in a whole-body magnetic resonance scan. The position of the sub-scan is the position of the scan area of each sub-scan performed on the subject P.

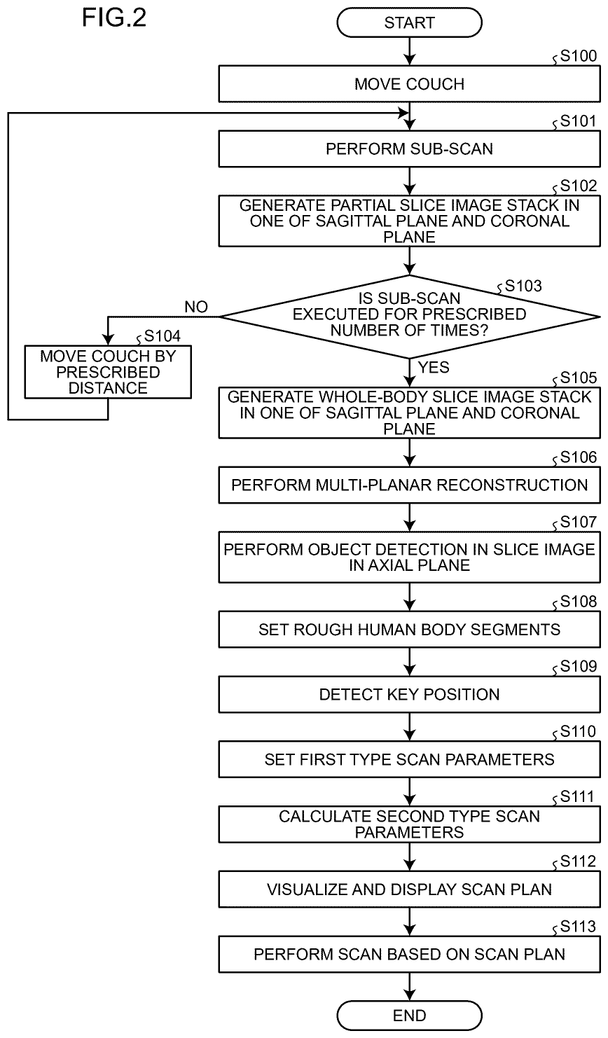

FIG. 2 is a flowchart illustrating the magnetic resonance imaging support method according to the first embodiment. The magnetic resonance imaging support method supports the user in generating a scan plan for a main scan based on a scout image. Hereinafter, the magnetic resonance imaging support method according to the present embodiment will be described by referring to FIG. 2.

At steps S100 to S105, a scout scan for generating scout images of the subject P is performed to acquire a whole-body slice image stack in either the sagittal planes or the coronal planes as the scout images. In a scout scan, a plurality of sub-scout scans covering the whole body of the subject P are performed in sequence. The range covered by the sub-scout scans is determined by setting the start position and end position of the scout scan. Each of the start position and end position of the scout scan may be set at the head top of the subject P and the sole of the foot of the subject P, for example.

At step S100, the couch control circuitry 105 moves the couch 104 with the subject P placed thereon such that the head top of the subject P is positioned in the scan area within the substantially cylindrical shaped gradient coil 102.

Note here that the subject P is positioned such that there is sufficient room between the head top and the boundary of the scan area in order to ensure that the scout scan covers the whole body of the subject P.

After completing step S100, the processing proceeds to step S101.

At step S101, the image processing unit 126 performs sub-scout scans to acquire partial slice images in either the sagittal planes or the coronal planes at a plurality of slice positions set in advance.

In the sub-scout scans, sequence information of the scan sequence for executing the sub-scout scans is transmitted to the sequence control circuitry 110, and the k-space data at the slice positions set in advance acquired by the sub-scout scans is received from the sequence control circuitry 110. Thereafter, the image reconstruction unit 125 performs a Fourier transform on the received k-space data to acquire partial slice images at the slice positions.

A partial slice image is two-dimensional image data, and the size thereof is determined by the FOV used for the scout scan. Each of the partial slice images indicates the structure within the local range at each of the slice positions of the subject P.

In the present embodiment, a scout scan is either a coronal scan for generating images in the coronal plane or a sagittal scan for generating images in the sagittal plane. When the scout scan is a coronal scan, the slice positions set in advance are positions on a plurality of coronal planes parallel to each other determined in accordance with the body size and slice gap in the front-and-rear direction of the subject P, and each slice position is set with a slice gap provided in the front-and-rear direction of the subject P. When the scout scan is a sagittal scan, the slice positions are positions on a plurality of sagittal planes parallel to each other determined in accordance with the body size and slice gap in the left-and-right direction of the subject P, and each slice position is set with a slice gap provided in the left-and-right direction of the subject P.

Since scout scans have low requirements in terms of the resolution, contrast, and the like, a wide FOV is employed to shorten the scan time, and a fast scan sequence such as gradient echo (GRE) or the like is selected as the scan sequence of the scout scan.

After completing step S101, the processing proceeds to step S102.

At step S102, the image processing unit 126 generates a partial slice image stack in either the sagittal planes or the coronal planes by sequentially arranging the partial slice images acquired at step S101. When the partial slice images are images in the coronal planes, a partial slice image stack is generated by arranging the partial slice images in the front-and-rear direction of the subject P. When the partial slice images are images in the sagittal planes, a partial slice image stack is generated by arranging the partial slice images in the left-and-right direction of the subject P.

A partial slice image stack is W×H×N three-dimensional image data, where W and H represent the width and the height of the partial slice image, respectively, and N represents the number of partial slice images. A partial slice image stack indicates the structure within a local range at a plurality of slice positions of the subject P.

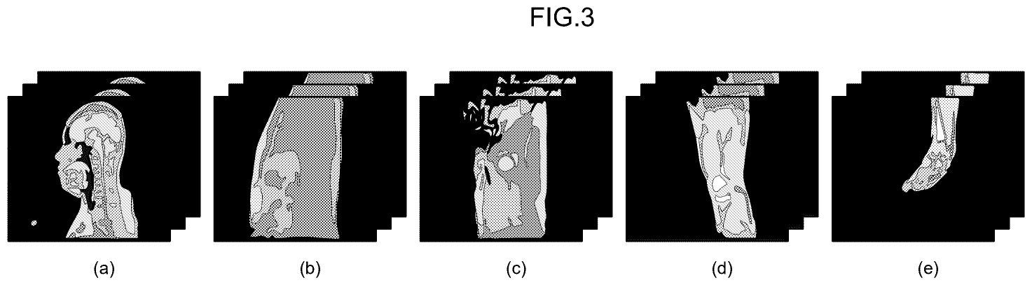

FIG. 3 is a diagram illustrating examples of partial slice image stacks. Note that (a) to (e) in FIG. 3 indicate partial slice image stacks in the sagittal planes regarding the head and neck, the chest and upper abdomen, the lower abdomen and buttock, the leg, and the ankle and foot of the subject P, respectively. Each partial slice image stack contains slice images in the sagittal planes at different slice positions regarding a specific part of the subject P.

The explanation continues by returning to FIG. 2. After completing step S102, the processing proceeds to step S103.

At step S103, the image processing unit 126 determines whether a prescribed number of sub-scout scans are executed and, proceeds to step S104 when determined that a prescribed number of times of sub-scout scans are not executed, while proceeding to step S105 when determined that a prescribed number of times of sub-scout scans are executed. The number of times of sub-scout scans is determined by the height (size in the top-and-bottom direction) of the subject P, the FOV to be used, and the couch moving distance. The number of times of sub-scout scans is set such that the range of the last sub-scout scan covers the end position of the scout scan.

At step S104, the couch control circuitry 105 moves the couch 104 by a prescribed couch moving distance to move the part of the subject P to be scanned next to the scan area of the magnetic resonance imaging apparatus 100. Here, for aligning the positions of the adjacent partial slice image stacks, it is preferable to set the couch moving distance such that there is a mutually overlapping part between the partial slice images adjacent to each other in the top-and-bottom direction of the subject P. After completing step S104, the processing proceeds to step S101.

At step S105, the image stitching unit 61 of the image processing unit 126 aligns the positions of the adjacent partial slice image stacks based on a plurality of partial slice images included in the partial slice image stacks, and combines the aligned partial slice image stacks together to generate a whole-body slice image stack in either the sagittal planes or the coronal planes.

A whole-body slice image stack in either the sagittal planes or the coronal planes contains a plurality of whole-body slice images in the corresponding sagittal planes or coronal planes arranged in sequence. The whole-body slice images indicate the structure within the whole body range at a plurality of slice positions within the subject P.

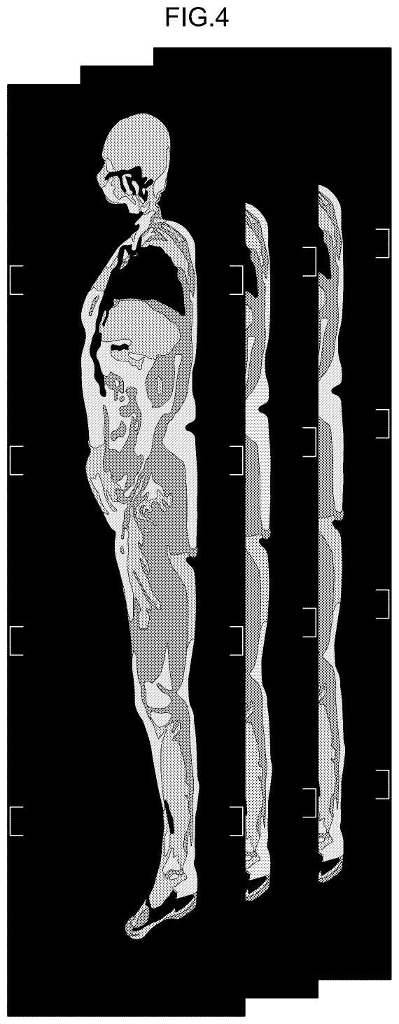

FIG. 4 is a diagram illustrating an example of the whole-body slice image stack. FIG. 4 illustrates a whole-body slice image stack in the sagittal planes acquired by combining together the partial slice image stacks of the subject P in the sagittal planes illustrated in FIG. 3.

The explanation continues by returning to FIG. 2. After completing step S105, the processing proceeds to step S106.

At step S106, the multi-planar reconstruction unit 62 of the image processing unit 126 performs multi-planar reconstruction based on the three-dimensional data contained in the whole-body slice image stack in either the sagittal planes or the coronal planes to generate a whole-body slice image stack in the axial planes and a whole-body slice image stack in the others of the sagittal planes and the coronal planes. The whole-body slice image stack in the others of the sagittal planes and the coronal planes contains a plurality of whole-body slice images in the corresponding sagittal planes or coronal planes arranged in sequence. The whole-body slice image stack in the axial planes contains a plurality of whole-body slice images in the axial planes arranged in sequence.

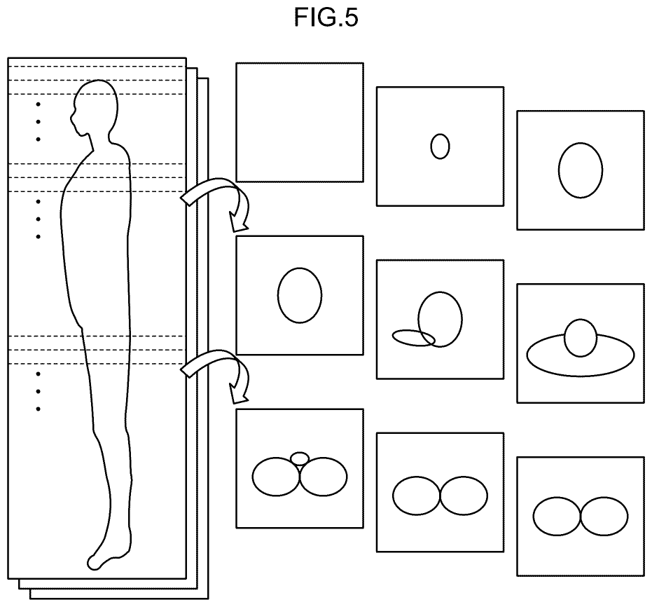

The processing of multi-planar reconstruction will be described below by referring to, as an example, a case of generating a whole-body slice image stack in the axial planes based on a whole-body slice image stack in the sagittal planes. FIG. 5 is a schematic diagram illustrating an example when generating whole-body slice images in axial planes based on a whole-body slice image stack in sagittal planes. As illustrated in FIG. 5, a plurality of reconstruction positions (positions indicated by the dotted lines in the drawing) that are apart from each other by a specific distance in the top-and-bottom direction of the subject P are set in the whole-body slice image stack in the sagittal planes, and the whole-body slice images are generated at the respective reconstruction positions by performing processing such as interpolation, resampling, projection, and the like on the whole-body slice images in the sagittal planes at each of the reconstruction positions. After generating the whole-body slice images in a plurality of axial planes, the whole-body slice images in the axial planes are arranged sequentially in the top-and-bottom direction of the subject P to generate a whole-body slice image stack in the axial planes.

In the present embodiment, not only the whole-body slice image stack in either the sagittal planes or the coronal planes acquired by the scout scan, but also the whole-body slice image stack in the axial planes generated by the multi-planar reconstruction and the whole-body slice image stack in the others of the sagittal planes and the coronal planes are set as the scout images.

The explanation continues by returning to FIG. 2. After completing step S106, the processing proceeds to step S107.

At steps S107 to S111, a scan plan for the main scan is generated based on the whole-body slice image stacks in the axial planes, the sagittal planes, and the coronal planes.

At step S107, the object detection unit 63 of the image processing unit 126 performs object detection on the whole-body slice image in each of the axial planes included in the whole-body slice image stacks in the axial planes using a trained neural network. Object detection is an important technology in computer vision, which is a technology that detects whether there is a target object in an image and marks the position of the target object in the image with a bounding box. In the present embodiment, three rough segments of the human body, which are the head, trunk, and lower limbs, are set as the detection targets. A neural network model used for object detection may be a feedforward neural network, a convolutional neural network, Transformer, or the like.

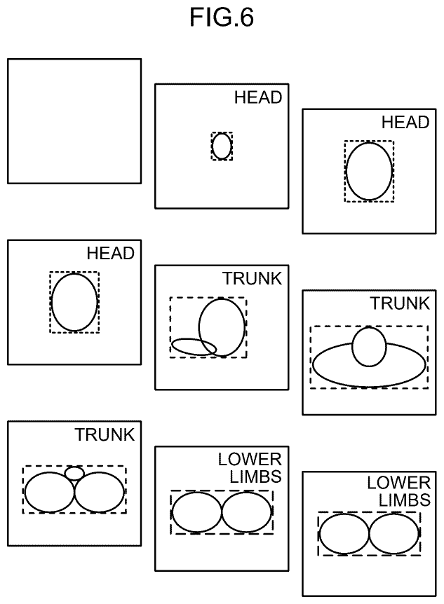

FIG. 6 is a schematic diagram illustrating a result of object detection performed on the whole-body slice images in the plurality of axial planes illustrated in FIG. 5. As illustrated in FIG. 6, the head, trunk, or lower limb is detected as a target object in each of the whole-body slice images in the axial planes, and the position of each target object is indicated by a bounding box.

The explanation continues by returning to FIG. 2. After completing step S107, the processing proceeds to step S108.

At step S108, the object detection unit 63 of the image processing unit 126 sets rough human body segments in the whole-body slice image stacks in the axial planes, the sagittal planes, and the coronal planes in accordance with the target object types detected in the whole-body slice image in each of the axial planes. Specifically, rough human body segments are set in the whole-body slice image stacks in the axial planes by using the types of the detection targets detected in the whole-body slice image in each of the axial planes as the rough human body segments where the axial plane exists. Then, the rough human body segments of the whole-body slice image stacks in the sagittal planes and the coronal planes are set by corresponding to the rough human body segments of the whole-body slice image stack in the axial planes.

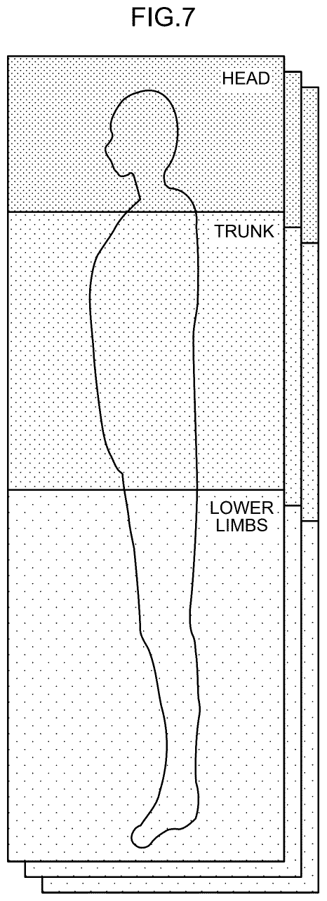

FIG. 7 is a schematic diagram illustrating rough human body segments in whole-body slice images in the sagittal planes, each whole-body slice image being set based on the whole-body slice images in the axial planes. As illustrated in FIG. 7, a whole-body slice image in the sagittal plane is sectioned (divided) into three areas that are the head, trunk, and lower limbs.

The explanation continues by returning to FIG. 2. After completing step S108, the processing proceeds to step S109.

At step S109, the key position detection unit 64 of the image processing unit 126 detects the key position regarding the subject P based on the whole-body slice image stacks in the axial planes, the sagittal planes, and the coronal planes as the scout images. Note here that the key position is a specific position of the human body, such as the head top, the head center, the spine, the knee center and the sole of the foot, or the like. The key position is used for determining the second type scan parameters such as the start position and end position of a whole-body magnetic resonance scan during the generation of the scan plan, the number of times of sub-scans, and the positions of the sub-scans, and the like in a scan plan. In the present embodiment, slice images related to a specific part of the subject P are selected from the whole-body slice image stacks in the sagittal planes and the coronal planes based on the whole-body slice image stack in the axial planes, a segment image indicating the rough human body segment where the specific part exists is cut out from the selected slice images, and the position of the specific part in the segment image is detected as the key position.

As examples, each of a method for specifying the positions of the head top and the head center, a method for specifying the position of the knee center, and a method for specifying the position of the spine will be described below.

### Specifying Positions of Head Top and Head Center

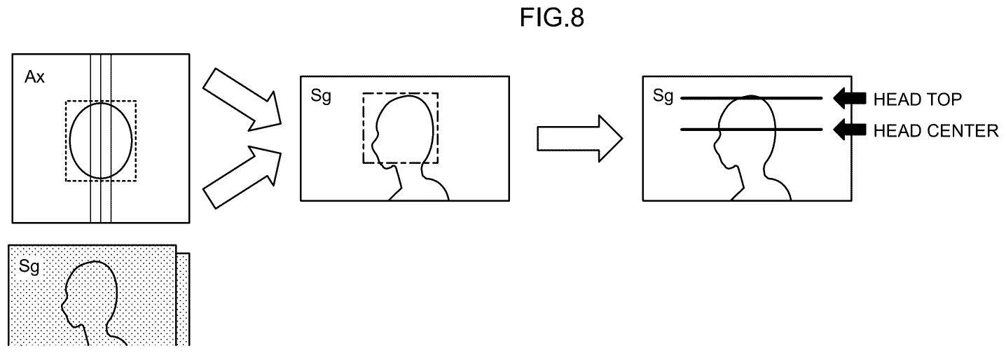

FIG. 8 is a schematic diagram illustrating a method for specifying the positions of the head top and the head center based on the whole-body slice image stacks in the axial planes and the sagittal planes. Referring to FIG. 8, the method for determining the positions of the head top and the head center based on the whole-body slice image stacks in the axial planes and the sagittal planes will be described.

First, in the head region of the whole-body slice image stack in the axial planes, the slice image with the largest head cross-sectional area is specified. The cross-sectional area of the head can be acquired by the size of the bounding box of the detected head. Note that it is not limited to the slice image with the largest head cross-sectional area. For example, in the head region of the whole-body slice image stack in the axial planes, a slice image in which the head cross-sectional area that is 90% of the largest head cross-sectional area may be specified.

Thereafter, one or more whole-body slice images in the sagittal planes closest to the head center in the slice image in the axial plane in the left-and-right direction of the subject P are selected. When selecting a plurality of whole-body slice images in the sagittal planes, for example, slice images within 2 cm from the head center may be selected.

Thereafter, a head image indicating the head segment is cut out from a selected single whole-body slice image in the sagittal plane or an average image of a plurality of whole-body slice images in the sagittal planes, objection detection is performed for the head in the cutout head image using a trained neural network, and the contour of the head is surrounded by a bounding box.

Then, the upper frame position of the bounding box of the detected head is defined as the position of the head top, and the position in the center of the top-and-bottom direction in the bounding box of the head is defined as the position of the head center.

### Specifying Position of Knee Center

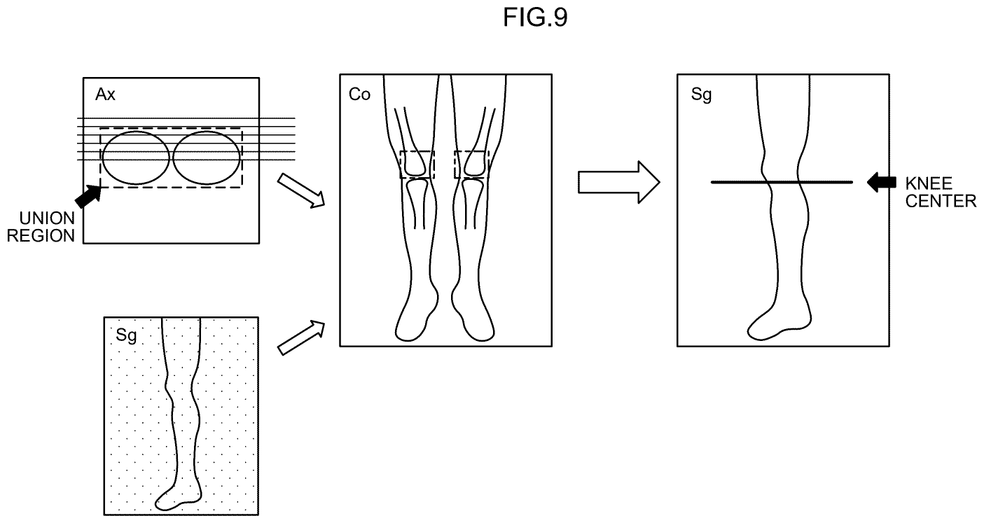

FIG. 9 is a schematic diagram illustrating the method for specifying the position of the knee center based on the whole-body slice image stacks in the axial planes and the coronal planes. Referring to FIG. 9, the method for specifying the knee center based on the whole-body slice image stacks in the axial planes and the coronal planes will be described.

First, in the upper half of the lower limb region of the whole-body slice image stack in the axial planes, the union of the regions surrounded by each of the bounding boxes is acquired for all of the whole-body slice images as a union region.

Then, in the front-and-rear direction of the subject P, one or more whole-body slice images in the coronal planes positioned at the anterior thigh in the acquired union region are selected.

Thereafter, a lower limb image indicating the lower limb segment is cut out from a selected single whole-body slice image in the coronal plane or an average image of a plurality of whole-body slice images in the coronal planes, objection detection is performed for the femur in the cutout lower limb image using a trained neural network, and the contour of the femur is surrounded by a bounding box.

Then, the lower frame position of the bounding box of the detected femur is defined as the position of the knee center.

### Specifying Position of Spine

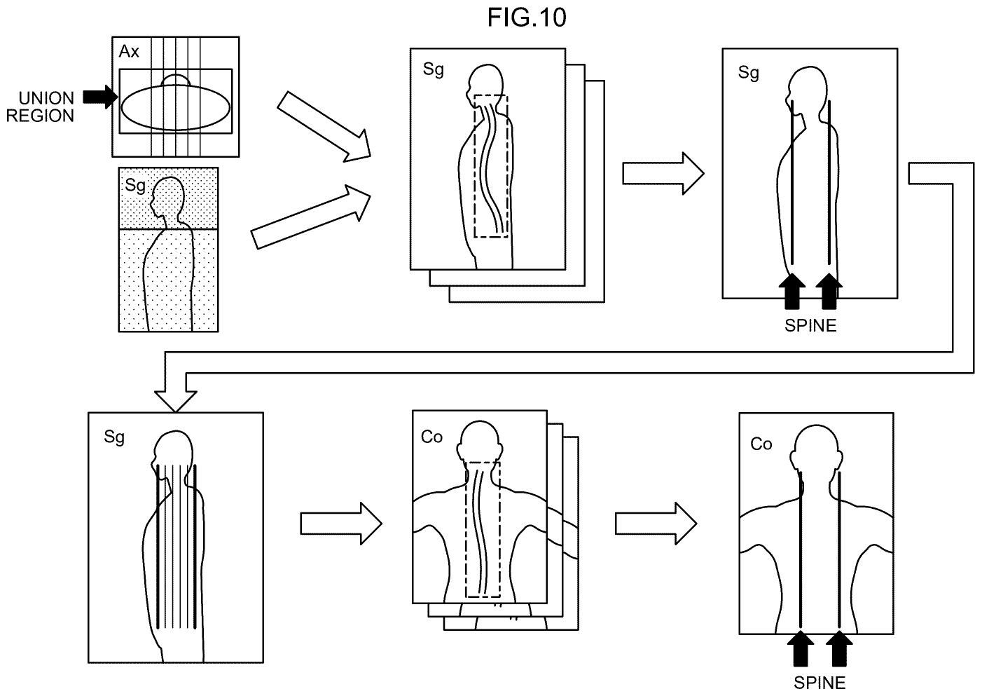

FIG. 10 is a schematic diagram illustrating a method for specifying the position of the spine based on the whole-body slice image stacks in the axial planes, the sagittal planes, and the coronal planes. Referring to FIG. 10, the method for specifying the position of the spine based on the whole-body slice image stacks in the axial planes, the coronal planes, and the sagittal planes will be described.

First, in the center part of the trunk segment of the whole-body slice image stack in the axial planes, the union of the regions surrounded by each of the bounding boxes is acquired as a union region for all of the whole-body slice images.

Then, in the left-and-right direction of the subject P, one or more whole-body slice images in the sagittal planes positioned in the center part of the trunk in the acquired union region are selected.

Thereafter, a head and trunk image indicating the head and trunk segment is cut out from a selected single whole-body slice image in the sagittal plane or an average image of a plurality of whole-body slice images in the sagittal planes, objection detection is performed for the spine in the cutout head and trunk image using a trained neural network, and the contour of the spine is surrounded by a bounding box.

Then, the left frame position, right frame position, upper frame position, and lower frame position of the bounding box of the spine detected in the sagittal plane are defined as the frontmost edge, rearmost edge, uppermost edge, and lowermost edge of the spine, respectively.

Then, in the sagittal plane, one or more whole-body slice images in the coronal planes positioned between the frontmost edge and the rearmost edge of the spine in the front-and-rear direction of the subject P are selected.

Thereafter, a head and trunk image indicating the head and trunk segment is cut out from a selected single whole-body slice image in the coronal plane or an average image of a plurality of whole-body slice images in the coronal planes, objection detection is performed for the spine in the cutout head and trunk image using a trained neural network, and the contour of the spine is surrounded by a bounding box.

Then, the left frame position of the bounding box of the spine detected in the coronal plane is defined as the left-most position of the spine, and the right frame position of the bounding box of the spine is defined as the right-most position of the spine.

The explanation continues by returning to FIG. 2. After completing step S109, the processing proceeds to step S110.

At step S110, via the input device, the user sets the first type scan parameters that include at least the inspection range of the main scan, the FOV, and couch moving distance. After completing step S110, the processing proceeds to step S111.

At step S111, the scan plan generation unit 127 generates a scan plan for the main scan by calculating at least one of the second type scan parameters that include the start position and end position of the main scan, the number of times of sub-scans, the positions of sub-scans, based on the inspection range, the FOV, and the couch moving distance set by the user at step S110 as well as the key position regarding the subject P specified at step S109.

Specifically, first, the start position and end position of the main scan are set based on the inspection range set by the user at step S110. For example, when the user sets the inspection range as the whole body, the position of the head top or the head center specified at step S109 is set as the start position of the main scan and the position of the knee center or the sole specified at step S109 is set as the end position of the main scan. Furthermore, when the user sets the inspection range as the spine, for example, the positions of the uppermost end and lowermost end of the spine specified at step S109 are set as the start position and end position of the main scan, respectively. That is, the range to be scanned in the main scan is set.

The number of sub-scans required to cover the start position and end position of the main scan is then calculated from the start position and end position of the main scan, the FOV size, and the couch moving distance. Specifically, the smallest m with which the sum of the FOV size and m times the couch moving distance becomes greater than the distance between the start position and end position of the main scan is calculated as the number of sub-scans.

Then, the scan positions in the top-and-bottom direction of the subject P for each of the sub-scans are determined such that the top end of the scan area of the first sub-scan coincides with the start position of the main scan and that the top end of the scan area of each of the sub-scans thereafter is spaced from the top end of the scan area of the previous sub-scan by the couch moving distance. Then, the scan positions in the left-and-right and front-and-rear directions of the subject P for each of the sub-scans are determined such that the mid-axis lines in the left-and-right and front-and-rear directions of each of the sub-scans coincide with the mid-axis lines of the body of the subject P. The mid-axis lines of the subject P can be specified by the key position such as the spine, for example.

Finally, a scan plan for the main scan is generated based on the first type scan parameters set by the user and the calculated second type scan parameters.

After completing step S111, the processing proceeds to step S112.

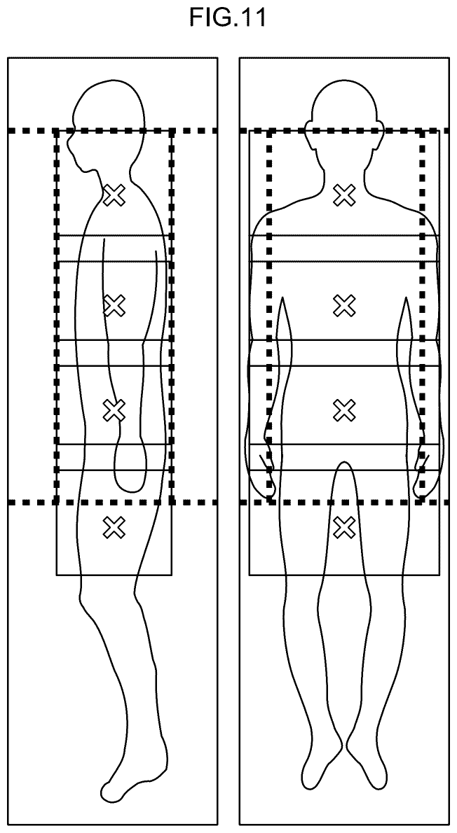

At step S112, the scan plan generation unit 127 visualizes the scan plan for the main scan and displays it on the display unit 122. FIG. 11 is a diagram illustrating an example of the scan plan for the main scan displayed on the display unit 122. In FIG. 11, the start position and end position of the main scan are indicated by dashed lines, and the positions of each of the sub-scans are indicated by solid lines.

After completing step S112, the processing proceeds to step S113.

At step S113, the sequence control circuitry 110 performs the scan on the subject P based on the scan plan for the main scan.

When the processing of step S113 is completed, the processing of the magnetic resonance imaging support method is finished.

### Effect of Embodiment

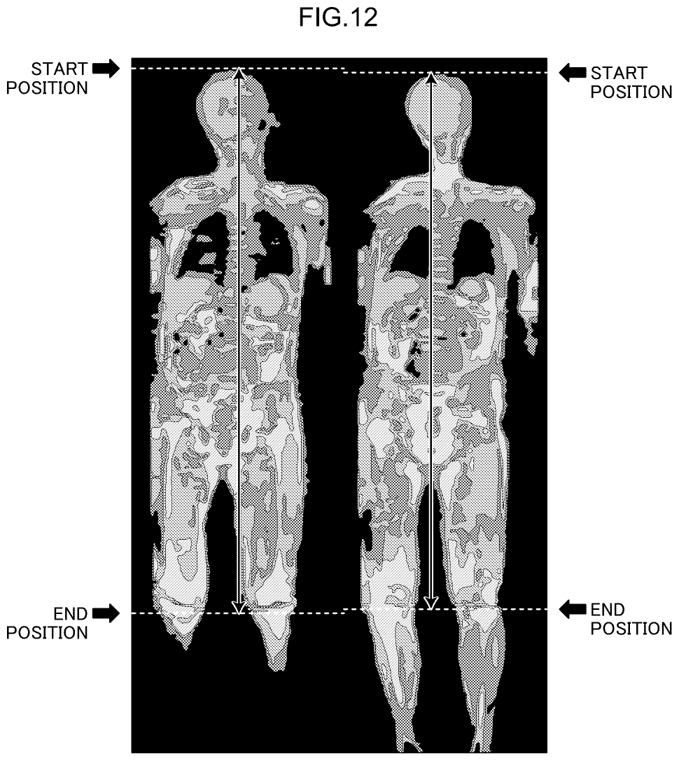

In conventional magnetic resonance imaging support methods, a single slice image is used as a scout image to generate a scan plan for the main scan, so that the scan plan comes to depend on the slice position of the scout image. FIG. 12 is a diagram for describing the issues of the conventional magnetic resonance imaging support methods. FIG. 12 indicates the start position and end position of the main scan specified by the scout images at two different slice positions. As can be seen from FIG. 12, start positions and end positions of different main scans may be specified depending on the scout images at different slice positions, which may result in specifying inaccurate scan positions.

According to the present embodiment, the start position and end position of the main scan and the positions of the sub-scans are specified based on the three-dimensional scout images in the axial plane, sagittal plane, and coronal plane. Therefore, it is possible to improve the accuracy of the specified start position and end position of the main scan as well as the positions of the sub-scans. In addition, according to the present embodiment, the start position and end position of the main scan and the positions of the sub-scans are specified based on the key position that is detected by selecting the appropriate slice image. Therefore, it is possible to further improve the accuracy of the specified start position and end position of the main scan as well as the positions the sub-scans.

Furthermore, according to the present embodiment, only about 1/20 of the slice images out of the whole-body slice image stacks in the axial planes, the sagittal planes, and the coronal planes as the scout images are used to generate a scan plan. Therefore, it is possible to reduce the amount of computation required to generate a scan plan.

According to the present embodiment, it is possible to support the generation of an appropriate scan plan for a main scan.

### Training of Deep Learning Model

In the above description, the object detection unit 63 of the image processing unit 126 according to the present embodiment uses a trained neural network to perform object detection in the slice images. Hereinafter, a training method of the neural network will be described.

When training of the neural network is started, first, a plurality of sets of training data stored in advance are read out from the storage unit 124. Each set of training data includes slice images in the axial planes, the sagittal planes, and the coronal planes as input data, and the bounding box and type of a target object as ground truth data.

Then, a plurality of sets of training data are divided into a training set and a test set. Examples of percentages of the training set and the test set may be 80% and 20%, or 90% and 10%. For example, when the total number of training data pieces is 10000 sets, the training data from data pieces #1 to #10000 are divided into data pieces #1 to #8000 as a training set and data pieces #8001 to #10000 as a test set. Then, by inputting input data in each set of training data in the training set to the neural network, estimating the bounding box and type of the detection target, calculating the difference value between the estimated value and the ground truth data, and performing back-propagation based on the difference value, the parameters of the neural network are changed such that the difference value between the estimated value and the ground truth output by the neural network becomes smaller. The above process is repeated until the difference value between the estimated value and the ground truth data output by the neural network becomes smaller than a threshold set in advance for most of the data in the test set. Thereafter, it is determined that training of the neural network is completed.

### Modification Example

The case of generating, based on a whole-body slice image stack in either the sagittal planes or the coronal planes, a whole-body slice image stack in the axial planes and a whole-body slice image stack in the others of the sagittal planes and the coronal planes is described above. However, when it is sufficient to specify the key position regarding the subject P based only on the whole-body slice image stack in the axial planes and the whole-body slice image stack in either the sagittal planes or the coronal planes, it is not necessary to generate the whole-body slice image stack in the others of the sagittal planes and the coronal planes. According to the modification example, it is possible to reduce the amount of computation of image processing.

### Second Embodiment

A second embodiment relates to a magnetic resonance imaging support method. Hereinafter, the magnetic resonance imaging support method according to the second embodiment will be described with reference to the accompanying drawings. In the second embodiment, the different points with respect to the first embodiment will mainly be described, and explanation of the points in common with the first embodiment will be omitted. In the description of the second embodiment, the same reference symbols are applied to the same components as those of the first embodiment.

The magnetic resonance imaging support method according to the second embodiment further includes step S114A and includes step S111A instead of step S111, compared to the first embodiment.

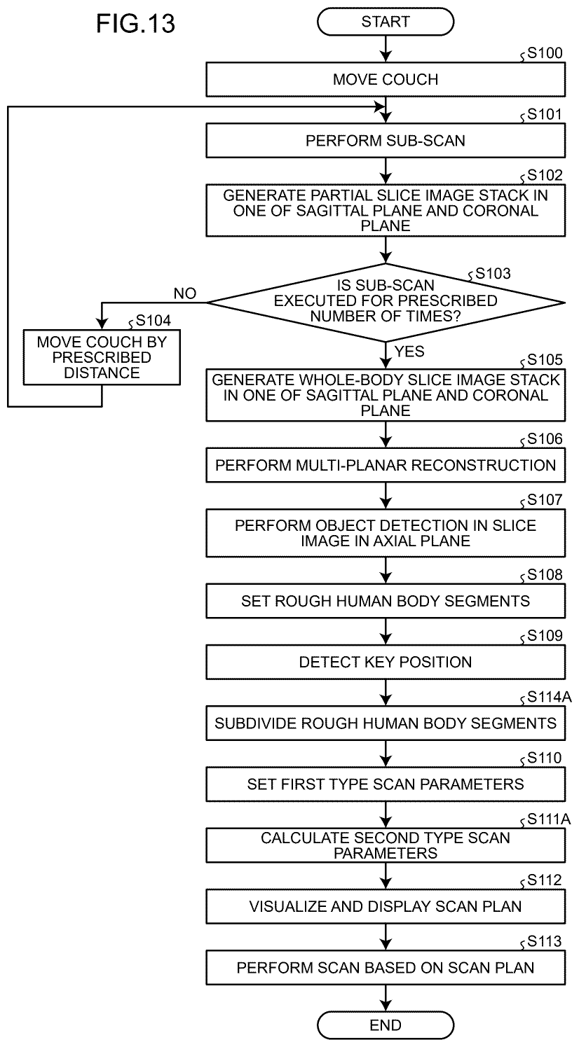

FIG. 13 is a flowchart illustrating the magnetic resonance imaging support method according to the second embodiment. Hereinafter, the magnetic resonance imaging support method according to the present embodiment will be described by referring to FIG. 13.

In the magnetic resonance imaging support method according to the present embodiment, the processing proceeds to step S114A when step S109 is completed.

At step S114A, the object detection unit 63 of the image processing unit 126 further subdivides the human body segment of the trunk into the thorax segment, abdomen segment, and pelvis segment based on the detected key positions, and further subdivides the human body segment of the lower limb into the leg segment and ankle segment. The thorax segment, abdomen segment, and pelvis segment may be sectioned, for example, by the positions of the heart and pelvis. The leg segment and the ankle segment may be sectioned by the position of the ankle, for example. After completing step S114A, the processing proceeds to step S110.

After completing step S110, the processing proceeds to step S111A.

At step S111A, the scan plan generation unit 127 calculates the start position of the main scan, the number of times of sub-scans, and the positions of the sub-scans based on the inspection range, the FOV, the couch moving distance, and the key position regarding the subject P and also calculates the specific absorption rate of each of the sub-scans from the subdivided human body segments covered by each of the sub-scans. The specific absorption rate is an important safety parameter in magnetic resonance imaging, and it represents the amount of RF energy absorbed by a unit mass of tissues in a unit time. The magnitude of the absorption rate directly affects the risk of thermal damage that may occur to the tissues of the body of the subject P during a magnetic resonance imaging inspection. The specific absorption rate needs be set for the subdivided human body segments.

In the embodiments described above, the image stitching unit 61, the multi-planar reconstruction unit 62, and the key position detection unit 64 of the image processing unit 126 are examples of the acquisition unit, the multi-planar reconstruction unit, and the first determination unit, respectively. The scan plan generation unit 127 is an example of the second determination unit. In addition, the sequence control circuitry 110 is an example of the main scan unit.

In the above embodiments, an example of the case is described in which the image stitching unit, the multi-planar reconstruction unit, the object detection unit, and the key position detection unit described herein are implemented by the image stitching unit 61, the multi-planar reconstruction unit 62, the object detection unit 63, and the key position detection unit 64 of the image processing unit 126, respectively. However, embodiments are not limited thereto. For example, other than implementing the image stitching unit, the multi-planar reconstruction unit, the object detection unit, and the key position detection unit described herein by the image stitching unit 61, the multi-planar reconstruction unit 62, the object detection unit 63, and the key position detection unit 64 described in the embodiments above, a processing unit having the same functions may be implemented by hardware only, software only, or a mixture of hardware and software.

In the embodiments described above, the couch control circuitry 105, the sequence control circuitry 110, the image processing unit 126, and the scan plan generation unit 127 are implemented by processing circuitry such as a processor, for example. In this case, each of the processing functions of the processing circuitry is stored in the storage unit 124 in the form of a computer program that can be executed by a computer, for example. Then, the processing circuitry reads out and executes the computer programs from the storage unit 124 to implement the processing functions corresponding to the computer programs. In other words, each circuit and each unit come to have the configurations illustrated in FIG. 1 in a state where the corresponding processing circuitry reads out the respective computer programs. While it is described herein that a single storage unit stores computer programs corresponding to the processing functions of the processing circuitry, the embodiments are not limited thereto. For example, the computer programs corresponding to each of the processing functions may be stored in a plurality of storage units in a distributed manner, and the processing circuitry may read out and execute each of the computer programs from the respective storage units.

Furthermore, while it is described in the above description that the couch control circuitry 105, the sequence control circuitry 110, the image processing unit 126, and the scan plan generation unit 127 are each implemented by a single piece of processing circuitry, the embodiments are not limited thereto. For example, each circuit and each unit may be configured with a combination of a plurality of pieces of independent processing circuitry and implement each of the processing functions by executing the computer programs with each piece of the processing circuitry. Furthermore, the processing functions of each circuit and each unit may be distributed or integrated into a single piece of or a plurality of pieces of processing circuitry as appropriate. Furthermore, the processing functions of each circuit and each unit may be implemented by a mixture of hardware such as circuitry and software.

The term “processor” used in the above description means, for example, a circuit such as a central processing unit (CPU), a graphics processing unit (GPU), or an application specific integrated circuit (ASIC) and a programmable logic device (for example, a simple programmable logic device (SPLD), a complex programmable logic device (CPLD), and a field programmable gate array (FPGA)). When the processor is a CPU, for example, the processor reads out and executes the computer program stored in the storage unit to implement the functions. In the meantime, when the processor is an ASIC, for example, instead of saving the computer program in the storage unit, the functions are directly incorporated as logic circuits within the circuitry of the processor. Note that each of the processors of the present embodiments is not limited to being configured as a single circuit for each processor, and may also be configured as a single processor by combining a plurality of independent circuits to implement the functions. Furthermore, it is also possible to integrate a plurality of structural components in FIG. 1 into a single processor to implement the functions.

Note here that the computer program to be executed by the processor is provided by being installed in advance in a read-only memory (ROM), a storage unit, or the like. The computer program may be provided in a file of format that can be installed on such devices or in an executable format by being recorded on a computer readable storage medium such as a compact disc (CD)-ROM, a flexible disk (FD), a CD-recordable (CD-R), a digital versatile disc (DVD), or the like. Furthermore, the computer program may also be stored on a computer connected to a network such as the Internet, and provided or distributed by being downloaded via the network. For example, the computer program is configured with modules including each of the functional units described above. As for the actual hardware, the CPU reads out and executes the computer program from a storage medium such as a ROM, so that each of the modules is loaded onto a main memory device and generated on the main memory device.

Furthermore, in the embodiments described above, each of the structural components of each of the illustrated devices is the functional concept and is not necessarily need to be physically configured as illustrated in the drawings. In other words, the specific forms of distribution and integration of the devices are not limited to those illustrated in the drawings, but all or some of them can be functionally or physically distributed or integrated in any unit in accordance with various kinds of load, use state, or the like. Furthermore, all or some of the processing functions performed by respective devices can be implemented by the CPU and the computer program that is analyzed and executed by the CPU, or may be implemented by hardware using wired logic.

Regarding the processing described in the above embodiments, all or several pieces of the processing described to be performed automatically can be performed manually, or all or several pieces of the processing described to be performed manually can be performed automatically using a known method. In addition to the above, the processing procedures, control procedures, specific names, and information including various kinds of data and parameters discussed in the description and drawings can be changed as appropriate, unless otherwise noted.

According to at least one of the embodiments described above, it is possible to support the generation of an appropriate scan plan for a main scan.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A magnetic resonance imaging support method comprising:
acquiring, by performing a scout scan on a subject, a plurality of first slice images corresponding to different slice positions, the plurality of first slice images corresponding to either sagittal planes or coronal planes;
generating a second slice image corresponding to an axial plane by performing multi-planar reconstruction based on the plurality of first slice images;
determining a position corresponding to a specific part of the subject based on the second slice image;
determining a range to be scanned in a main scan based on the plurality of first slice images at the determined position; and
performing a scan on the subject in the determined range.

2. The magnetic resonance imaging support method according to claim 1, wherein the determining the range to be scanned in the main scan includes determining a start position and an end position of the scan in the main scan.

3. The magnetic resonance imaging support method according to claim 1, wherein the determining the range to be scanned in the main scan includes determining at least one selected from the number of and positions of a plurality of sub-scans included in the main scan.

4. The magnetic resonance imaging support method according to claim 3, wherein the determining the range to be scanned in the main scan includes determining includes determining at least one selected from the number of and the positions of the plurality of sub-scans based on a size of a set field of view (FOV).

5. The magnetic resonance imaging support method according to claim 1, wherein
the generating the second slice image includes generating a plurality of the second slice images, and
determining the position corresponding to the specific part of the subject includes determining the position corresponding to the specific part of the subject based on the plurality of the second slice images.

6. The magnetic resonance imaging support method according to claim 1, wherein the determining the range to be scanned in the main scan includes determining the range to be scanned in the main scan based on a position of a head top or a head center in the first slice image corresponding to the sagittal plane at the determined position.

7. The magnetic resonance imaging support method according to claim 1, wherein the determining the range to be scanned in the main scan includes determining the range to be scanned in the main scan based on a position of a knee center in the first slice image corresponding to the coronal plane at the determined position.

8. The magnetic resonance imaging support method according to claim 1, wherein the determining the range to be scanned in the main scan includes determining the range to be scanned in the main scan based on a position of spine in the first slice image corresponding to at least one of the sagittal plane and the coronal plane at the determined position.

9. A magnetic resonance imaging apparatus, comprising:
processing circuitry configured to
acquire, by performing a scout scan on a subject, a plurality of first slice images corresponding to different slice positions, the plurality of first slice images corresponding to either sagittal planes or coronal planes;
generate a second slice image corresponding to an axial plane by performing multi-planar reconstruction based on the plurality of first slice images;
determine a position corresponding to a specific part of the subject based on the second slice image;
determine a range to be scanned in a main scan based on the plurality of first slice images at the determined position; and
perform a scan on the subject in the determined range.
